(19) United States
(12) Patent Application Publication
(10) Pub. No.: US 2025/0046426 A1
(43) Pub. Date: Feb. 6, 2025

(54) Artificial intelligence-based cognitive-coping therapy (CCT) method, system, and application

(71) Applicants: Xianzhang Hu, North Potomac, MD (US); Rose Hu, North Potomac, MD (US); Bing Li, North Potomac, MD (US)

(72) Inventors: Xianzhang Hu, North Potomac, MD (US); Rose Hu, North Potomac, MD (US); Bing Li, North Potomac, MD (US)

(21) Appl. No.: 18/437,140

(22) Filed: Feb. 8, 2024

Related U.S. Application Data
(60) Provisional application No. 63/513,613, filed on Jul. 14, 2023.

(51) Int. Cl. G16H 20/70; G06F 40/20; G16H 40/67

(52) U.S. Cl. CPC G16H 20/70; G06F 40/20; G16H 40/67

(57) ABSTRACT

This invention pertains to a cognitive-coping therapy (CCT) method, system, and application that is a computer-implemented method for implementing AI-based CCT for a patient using natural language processing (NLP) and/or human-machine dialogue with/without an avatar. Based on the cognitive psychology and stress-coping theory, the CCT alleviates the excessive fear of negative event(s) and can be used to address mental disorders/conditions like obsessive-compulsive disorder (OCD), anxiety disorders (e.g., panic disorder, social phobia), and more. The AI system uses the NLP to comprehend the patient's symptoms and provide relevant interventions. Additionally, the system can monitor the patient's progress over time and adapt the interventions accordingly.

### FIELD OF THE INVENTION

Here, we present a method, system, and application for cognitive-coping therapy (CCT), a new psychotherapy aiming at the fear of negative/adverse events and using coping strategies to reduce the stress of symptoms. The method, system, and application are a computer-implemented method for implementing AI-based cognitive-coping therapy (CCT) for a patient using natural language processing (NLP), Artificial Intelligence Language Model (AILM) tools, and other generative AI products, and/or human-machine dialogue with/without an avatar, to treat mental disorders/conditions such as obsessive-compulsive disorder, and anxiety disorders (including panic disorder and social phobia), and other related conditions.

### BACKGROUND

Cognitive-coping therapy (CCT) for the treatment of obsessive-compulsive disorder, developed by the inventors based on their novel etiology hypothesis, is a new type of psychotherapy that is highly effective in treating mental disorders including but not limited to obsessive-compulsive disorder and anxiety disorders (especially social phobia and panic attacks). In CCT, fear of negative events (e.g., fear of contamination, fear that might harm oneself or others), obsessions, and compulsions are considered stressors. The effects of these stressors on compulsions will be reduced if they are properly coped with. CCT has been reported to achieve higher response and remission rates, lower relapse rate and drop-off rate in a 12-month follow-up, and higher levels of social-occupational function. CCT has similar efficacy in drug-resistant vs. non-drug-resistant OCD, and overt compulsions vs. covert compulsions. A previous study reports that patients with OCD who achieve remission showed decreased functional connectivity between the left amygdala and certain brain regions including the right cingulate, left superior parietal, and left inferior parietal lobe.

As used herein, “traditional therapy” is defined by the following case study steps for a patient dealing with a disorder:

The demand for mental health services is increasing worldwide, but unfortunately, there is a massive shortage of mental health specialists to meet these needs, particularly in humanitarian emergencies, low-income countries, and other areas with limited resources. To bridge this gap, non-specialists like lay health workers, teachers, social workers, and peer mentors have become essential resources for providing mental health services. However, current strategies demand substantial training and supervision, although this approach can be effective. They also require highly standardized interventions, which may paradoxically limit more person-centered treatment.

In episodic care, a patient has not seen a doctor for treatment for many months (even many years, especially in OCD patients), yet the patient has many thoughts and symptoms that are not captured or the doctor must ask about and record using the rudimentary pen and paper technique.

The patient must call the doctor's office, wait on the phone, and get an appointment typically weeks after the call.

As per HIPAA laws, the patient is required to complete multiple forms. Surveys and questionnaires are also filled out to employ evidence-based screening tools and eliminate the possibility of other disorders like schizophrenia. This helps in diagnosing and treating the underlying cause of the visit, such as OCD, anxiety disorder, or other mental disorders.

The patient must wait a long time in a waiting room.

The patient is seen by a nurse or doctor to get his/her vital signs.

The doctor finally meets the patient and starts by asking questions to get qualitative and objective measures.

The doctor asks the patient to review surveys/questionnaires and then needs time to score the answers.

The patient is being treated with a CCT method that is module-based. This means that therapy sessions typically occur weekly or every two weeks and can take place in person, over the phone, or through a web portal. However, this approach can be challenging for patients as it requires them to visit the office frequently for face-to-face therapy or to participate in phone or Internet-based CCT if they have broadband access. It places a significant burden on the patient and it is time-consuming for healthcare providers, including doctors, nurses, and psychologists.

The patient wishes to accelerate the program, rather than have it drag on for several weeks.

Sometimes patients feel lost and need guidance or support on days when they are unable to see their healthcare providers. They may struggle to communicate important information to their care coordination team, which includes their doctor, nurse, psychologist, and physical therapist.

It is evident that the conventional therapy approach is insufficient, time-consuming, and doesn't allow patients to hasten their CCT program. Hence, there is a requirement for a more comprehensive and adaptable CCT program that caters to the patient's necessities, beginning from the initial diagnosis to the treatment plan. Additionally, wearable and other portable monitoring devices can be utilized to automatically gather data and assess the patient's health and treatment status.

The AI-based CCT comprises a computer system that uses NLP, Artificial Intelligence Language Model (AILM) tools, or other generative AI products, and/or human-machine dialogue with/without an avatar to understand the patient's symptoms and provide appropriate interventions. The system comprises a user interface that allows the patient to interact with the system and a backend AI engine that processes the patient's inputs and provides personalized interventions. The system can provide a range of interventions, including psychoeducation, cognitive restructuring, attribution to a reason for the patient's symptoms, identifying the symptoms as stressful events, teaching the patient to use appropriate coping strategies, and fulfilling the patient's family, social, and occupational function.

The system starts by conducting an initial assessment of the patient's symptoms using a series of questions designed to identify the patient's obsessions, compulsions, anxiety, and panic attack. Based on the assessment, the system provides personalized interventions aimed at reducing the patient's symptoms. The system uses a combination of psychoeducation, cognitive restructuring, attributing the symptoms to a psychological factor, and stress/coping therapy to help the patient understand the nature of OCD, anxiety disorders, or other mental disorders, and develop coping strategies.

The system is designed to be adaptive, meaning that it can monitor the patient's progress over time and adapt the interventions accordingly. The system uses machine learning algorithms to analyze the patient's responses and determine which interventions are most effective. The system can also adjust the level of difficulty of the interventions based on the patient's progress.

In one embodiment of the invention, the system is integrated with a wearable device that monitors the patient's physiological responses (e.g., heart rate, skin conductance) during exposure and response prevention interventions. The system uses this information to provide real-time feedback to the patient and remind the patient to choose an appropriate coping strategy.

In another embodiment of the invention, the system is designed to be used in conjunction with a therapist. The system provides the therapist with real-time data on the patient's progress, allowing the therapist to monitor the patient's response to interventions and adjust the treatment plan as needed.

### CONCLUSION

The AI-based CCT provides an automated, personalized, and adaptive intervention for individuals with OCD, anxiety disorder, and other mental disorders. The system is designed to be user-friendly and accessible, making it a promising tool for providing affordable and scalable mental health interventions. The system has the potential to revolutionize the way OCD, anxiety disorder, and other mental disorders are treated, providing individuals with access to effective interventions regardless of their location or financial resources.

### SUMMARY

Embodiments disclosed herein combine CCT with the full, dynamic features of a device (e.g., smartphone, tablet, or computer).

The principles disclosed herein provide an alternative and much better AI-based CCT method than the traditional therapy experience discussed above. The principles disclosed herein are designed to allow a potential patient the ability to obtain AI-based CCT for a disorder/condition using a device (i.e., his/her smartphone, tablet, or computer) running an application comprising the disclosed principles. For example, appointments can be scheduled using a mobile device via the disclosed application. The application will allow the patient to fill out online surveys and other forms to provide an online pre-screening of the patient's reason for a visit or CCT (thus, using more of the face time with a doctor for treatment rather than paperwork). Wearable devices implementing health monitoring sensors could be used to feed the patient's vital signs to the application and thus wirelessly to the doctor or nurse (saving time). Once a CCT program is initiated, the application can guide the patient through the CCT and record the progress, to name a few features of the disclosed principles.

In one embodiment, an AI-based computer-implemented method using NLP and/or Artificial Intelligence Language Model (AILM) tools or other generative AI products for implementing CCT for a patient is provided. The method comprises inputting a patient profile via an application executing on a processor of a patient device, said application being adapted to input data directly from the patient and from one or more wearable electronic devices associated with the patient; analyzing the profile to generate a CCT plan for the patient; monitoring patient data and other input data obtained as the patient implements the CCT plan to determine a status of the CCT plan; and determining if the CCT requires adjustment based on the monitored patient data and other input data.

In another embodiment, a system for implementing CCT for treating a patient is provided. The system comprises an application program to be executed by a processor of a patient device, said application being adapted to: input a patient profile by inputting data directly from the patient and one or more wearable electronic devices associated with the patient; analyze the profile to generate a CCT plan for the patient; monitor patient data and other input data obtained as the patient implements the CCT plan to determine a status of the CCT plan; and determine if the CCT requires adjustment based on the monitored patient data and other input data.

### BRIEF DESCRIPTION OF THE DRAWINGS

FIG. 1 illustrates an example processing flow performed by an embodiment disclosed herein.

FIG. 2 illustrates an example processing flow performed by an embodiment used for CCT.

FIG. 3 illustrates an example system for implementing the disclosed application and method.

FIG. 4 illustrates the procedure of CCT for the treatment of OCD and anxiety disorders.

FIG. 5A Example screenshots of functionality provided by the disclosed CCT.

FIG. 5B illustrates example screenshots of treatment for OCD provided by the disclosed CCT.

### DETAILED DESCRIPTION

In this disclosure, the term patient, user, or client may be used interchangeably as a person or persons seeking treatment for a disorder/condition or disease that can be treated by a CCT program.

Accordingly, in one embodiment, the principles disclosed herein can provide a flexible or personalized therapy/treatment for a patient's disorder/condition via an application running on the patient's mobile device. The application is evidence-based (i.e., proven in the clinical world with efficacy and/or effectiveness data). To build such an application, in one embodiment, booklets/programs that are used in traditional face-to-face sessions/hospitals/clinics are converted and stored in one or more databases accessible by or stored on the mobile device. In one embodiment, an information architect is used to interview the clinician and maintain the integrity of the treatment program while also keeping the special healthcare “tone and voice” It should be appreciated that the format of a graphical user interface operated by the application is a complicated issue because of the device's small screen size. Moreover, the application will be able to communicate with a mobile health cloud system so a care coordination team can monitor and track the patient's treatment, and be a risk-detection platform.

The disclosed principles will convert the traditional therapy into a simple and more enjoyable experience that will allow patients a better opportunity to obtain and follow through on a prescribed therapy:

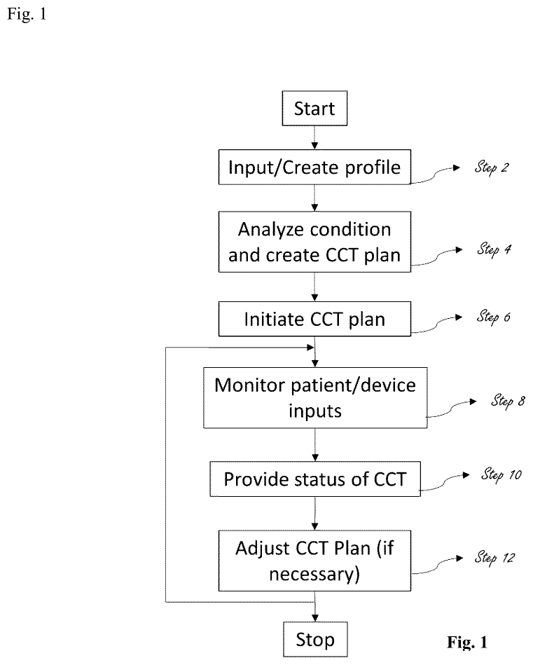

In one example embodiment, and reference to FIG. 1, a patient uses the disclosed application or activates the application by going through its different sections to perform CCT processing as follows.

The patient fills out a profile section on the application so that that application captures a baseline and rules out other diseases/disorders/conditions not related to the patient's current disease/disorder/condition (step 2). This can be a questionnaire provided by the application that is already established and used by the clinic (digitized and available to the mobile device), which can be scored based on the patient's answers. Alternatively, or in addition, the application can co-create with the patient a plan on how to treat the patient's disorder, etc. (e.g., stop smoking). For a smoker wishing to stop smoking, a profile can be used to determine the type of smoker the patient is and to set up a preliminary goal/plan to cease smoking. Accordingly, based on the patient's input information and/or information input by one or more devices connected to or worn by the patient, process analyzes the patient's condition (step 4). In addition, the disclosed principles can create a CCT plan that gathers the results of activities and questionnaires into a personalized plan. At this point, the patient's CCT is initiated (step 6).

At this point, the disclosed application can monitor inputs from the patient and/or one or more devices connected to or worn by the patient (step 8). The patients can track their daily activities (e.g., work, exercise, sleep, hobbies, etc.) by using a calendar function on the mobile device, which can be input into the disclosed CCT application.

The disclosed CCT application can allow the patient to journal and write narrative comments (e.g., the patient can use his/her phone to capture behaviors and events that may influence the therapy/disease (e.g., OCD, anxiety)). The logs are rich in data so that the application can calculate key metrics and create visualizations from them. The logs can track the patient's CCT progress. The patient can use the mobile device's camera to take pictures of his/her health condition. The logs can capture when a patient “slips” or has a “trigger” of its bad behavior—the disclosed CCT application provides easy access to the patient so that he/she can note triggers, cravings, etc. so the application can tailor a personalized plan (e.g., an individualized quitting plan for an OCD). With this information, the process can provide the patient with the status and progress of the CCT (step 10). Changes to the CCT plan can be made by the patient and/or application if needed (step 12). The process continues at step 8 until the CCT has been completed.

In one embodiment, the disclosed CCT application will have several coping strategy training sections that educate the patient about enhancing behaviors. The application can set goals and/or rewards for behavior changes.

In one embodiment, the disclosed CCT application will have a “cognitive” section that addresses unhelpful thinking. For example, the CCT application advises the patient of the reason(s) for the onset of OCD, social phobia, or panic disorder and how to cope with the reason(s); patients can create coping responses for challenging situations and get reminders when a tough event appears.

In one embodiment, the disclosed CCT application will have a “maintenance” section that creates a personalized reference and provides tips for handling lapses. For example, the disclosed CCT application can monitor the patient and keep him/her on track and can offer tips and techniques for preventing patients from relapse of symptoms.

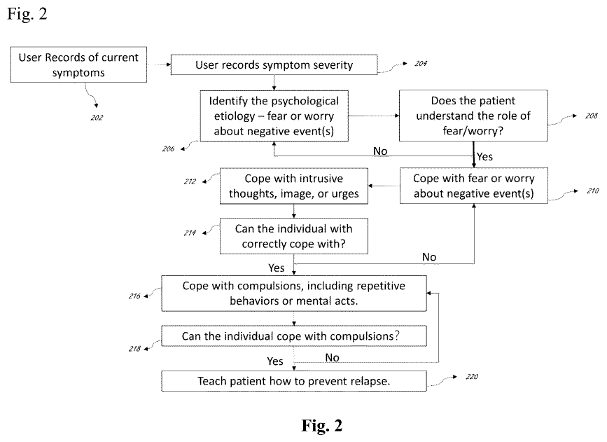

As shown in FIG. 2, the disclosed CCT application can implement CCT for OCD. Based on data gathered by one or more processors through user and/or sensor input (step 202) and the severity of symptoms (step 204), the CCT application can automatically determine appropriate CCT to address the user's specific OCD symptoms and modify CCT based on feedback obtained while therapy is ongoing (e.g., steps 6-12 of FIG. 1). For example, the CCT application may help users identify (step 206) and cope with the fear of negative events, obsessions, and urges to perform behavioral and/or mental rituals, using an artificial intelligence process. The CCT application may analyze the severity of OCD symptoms and the time that the patients spend on compulsions each day.

CCT application may update the CCT based on feedback. For example, the CCT application may receive a user-entered answer of ‘Yes’ or ‘No’ for a question (Do you understand the role of fear/worry in your problem?) (step 208) and determine whether the user goes to the nest step (step 210) or go back to the previous step (step 206).

The example processing from steps 206 to 220 of FIG. 2 illustrates a possible use case and therapy paths for the CCT application. After identifying a fear or worry about negative events, CCT will teach users to cope with the fear or the worry (step 210), followed by coping with obsessions (step 212). If users can cope with obsessions correctly (step 214), CCT will teach them to cope with compulsions (step 216); or go back to step 210. When users successfully cope with compulsions (step 218), the CCT application will teach users to prevent relapse (step 220); or go back to step 216. However, the CCT application is flexible enough to select, monitor, and modify any type of CCT. In some cases, a single user may use the CCT application to receive CCT for multiple maladies at the same time, for example. The CCT application provides a platform that can integrate any number of CCT courses into a single interface for a user.

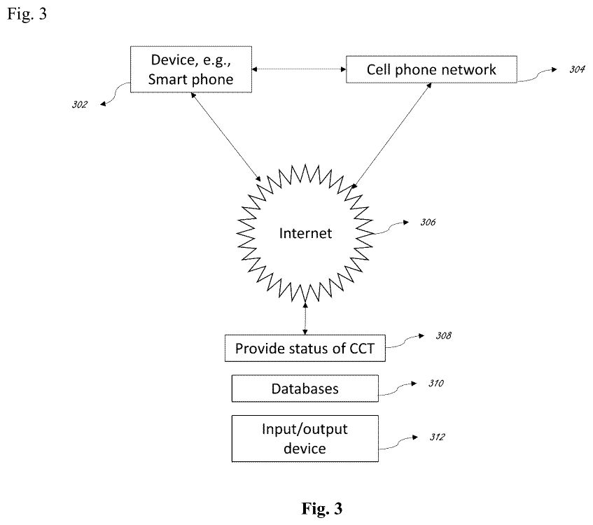

In one embodiment, the above-described method, system, and application are implemented in software (i.e., computer instructions) that are stored in a computer-readable memory and executed by a processor on both a patient device and a system server. FIG. 3 illustrates an example system comprising a CCT server (308) for communicating with a patient's mobile device (302) to implement the principles disclosed herein. The Server (308) includes or is connected to a memory (310) for storing computer instructions required to implement portions of the methods described herein and to store the various databases, user information, and login/account data used during the above-described processes. The system includes a database, which may also be stored in memory (312), for user accounts, CCT pamphlets, and clinical information, among other information required by the methods or applications disclosed herein. Server (308) can be accessed over a wired or wireless network (306) (shown as the Internet in this example) or via a cellular network (304).

Patient devices 302 include a mobile device (e.g., smartphone, tablet) that connects to server 308 via the Internet/network 306 and/or cellular network 304. Device 302 will also include a processor, memory, input/output components, and other devices (e.g., camera. GPS, accelerometer, etc.) that are useful for inputting and transmitting data disclosed herein. Although not shown, the system can also receive inputs from one or more devices connected to or worn by the patient (e.g., health monitors, exercise monitors, sleep monitors, sleep apnea sensors/monitors, breathing sensors, and heart rate monitors).

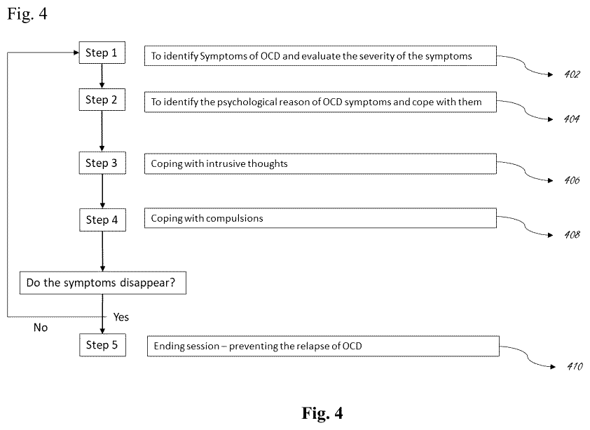

FIG. 4 illustrates the CCT procedure for treating OCD, anxiety disorders, and other mental disorders. In each session, there are four steps.

In step 1 (402), CCT aims to identify symptoms of OCD and evaluate the symptom severity. In this step, the OCD symptom checklist will be shown to users. To evaluate the severity of OCD symptoms, the Yale-Brown Obsessive-Compulsive Scale can be used. In addition, the index of OCD symptom severity is the method easily used for patients. There are several tools ready to be used. For example:

1. Clinical Global Impairment Scale: The CGIS requires users to consider their total clinical experience with their problem, and how mentally ill is the patient at the time. Circle one (most appropriate) of the following: 1=Normal, not at all ill; 2=Borderline mentally ill; 3=Mildly ill; 4=Moderately ill; 5=Markedly ill; 6=Severely ill; 7=Among the most extremely ill.

2. NIMH Global Obsessive-Compulsive Scale: Directions: Circle the number (1 to 15) that best describes the present clinical state of the patient based on the guidelines below.

On the other hand, users can use the Clinical Global Improvement Scale to evaluate how much he/she has changed: 1=Very much improved; 2=Much improved; 3=Minimally improved; 4=No change; 5=minimally worse; 6=Much worse; 7=Very much worse.

The aim of step 2 (404) is to help patients recognize and cope with fear. The first goal of this step was to help patients identify their fear and its role in the onset of OCD. In CCT, fear is considered an important factor in the onset of OCD. In essence, OCD patients are excessively afraid that negative events (e.g., death) will happen to them or their loved ones. It is the fear of imagined, dreaded negative events that invokes anxiety and results in neutralizing or avoidance behaviors (compulsions). Understanding the role of fear in the onset of OCD will help patients get ready to cope with fear. The second goal is to reduce the extent of fear, using appraisal-focused coping strategies (e.g., rational or denial) to cope with imagined negative events.

In step 3 (406), CCT aims to teach patients to cope with intrusive thoughts. First, it is necessary to identify the roles of intrusive thoughts in the causation of OCD. In CCT, intrusive thoughts symbolized the imaged, dreaded negative event the OCD patient feared. Second, using cognitive reconstruction, CCT helps the patients recognize there is no association between intrusive thoughts and negative events. Third, CCT will encourage patients to cope with intrusive thoughts by using appraisal-focused coping strategies, such as acceptance, ignorance, and/or sublimation. The goal is to teach patients to allow intrusive thoughts to exist in their minds, pay no attention to them, ignore them, and experience meaningful daily activities by practicing proper coping strategies.

In step 4 (408), CCT aims to teach the patients to cope with compulsions. The goal of this step was to eliminate compulsions. When steps 2 and 3 were completed successfully, patients would understand it is unnecessary to neutralize the fear, anxiety, or obsessions. Thus, it will be easier for patients to cope with and avoid the compulsions. Patients will be encouraged to practice using proper coping strategies in the therapy room. During this process, it was very important to avoid using ERP.

In each therapy session, patients moved through all four steps. When the OCD symptoms were eliminated after several sessions, the patient was habitually urged to check if the intrusive thoughts were still in their mind, which generally resulted in the immediate emergence of intrusive thoughts. To prevent relapse, the therapist reminded the patient that the urge to check should be considered an intrusive thought to be coped with.

Step 5 (410). Ending session—preventing the relapse of OCD. The aims of this step are 1. Inform patients never to check if the obsessions go away from their minds and 2. Inform patients never to check if they can control their obsessions and compulsions.

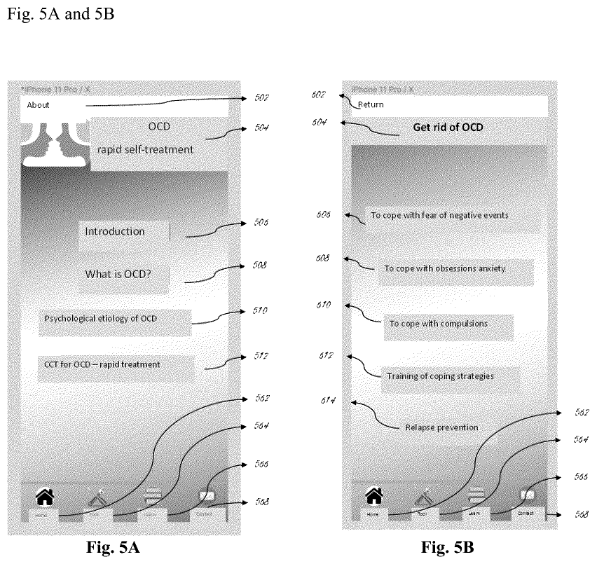

FIG. 5A and FIG. 5B illustrate example screenshots and functionality of pages provided on the patient's mobile device and by the disclosed CCT application when used for OCD in accordance with the disclosed principles. In the first example page FIG. 5A, a CCT for OCD worksheet is displayed on the device. The worksheet includes function button (502), page title (504), the introduction of CCT for OCD (506), basic information about OCD (508), the novel psychological etiology of OCD (510), the treatment procedure for OCD (512), and avatar human-machine dialogue (510). In addition, there are some functional buttons to guide users to go back homepage (562), tools (OCD symptoms and its evaluation) (564), learning material (566), and contact information (568).

In the second example page FIG. 5B, a CCT for OCD worksheet is displayed on the device. The worksheet includes function button (602), page title (604), coping with the fear of negative events (606), coping with obsessions and anxiety (608), coping with compulsions (610), training of coping strategies (612), and relapse prevention (614). In addition, there are some functional buttons to guide users to go back homepage (562), tools (OCD symptoms and its evaluation) (564), learning material (566), and contact information (568).

Accordingly, the principles disclosed herein translate an evidence-based CCT for OCD program into the disclosed and AI-based CCT application. CCT is medically proven to be superior to drugs in treating OCD. The disclosed CCT application can be used in conjunction with a healthcare provider or alone. The Application calculates key metrics and creates visualizations based on the logs Features of the disclosed CCT application for treating OC) include (1) Identifying OCD symptoms and evaluating the symptom severity; (2) Identifying and coping with fear; (3) Coping with obsessions (intrusive thoughts); (4) Coping with compulsions: several sessions will be used to train and teach the patients to cope with the fear of negative events and/or anxiety, obsessions, and compulsions; and (5) one to three sections teach the patient how to prevent from relapse. The CCT application leverages the interactivity of e-health solutions, including questionnaires, and checklists, as well as easy access to key content and tools, and a bookshelf for reference material; incorporates reminders and encouraging messaging.

The disclosed CCT method, system, and application will work as a substitute for CCT-OCD and social phobia therapy with a human therapist. The disclosed principles present the therapy as a stepped process, in much the same way a therapist would. There is a beginning, middle, and end phase to the therapy. This means the patient experiences the therapy in stages.

Typical CCT systems and methods prompt the users with a list of prompts that a patient rewards and looks back at their personalization. In sum, the disclosed embodiments combine both linear and non-linear presentations for maximum usefulness of a therapeutic application, which allows each user to move through the therapy at their own pace.

The special/different/novel/non-obvious about the disclosed embodiments include the below: The disclosed embodiments present CCT in a progression of sessions, much like in-person CCT therapy does. They are flexible and allow the users to be in control and take sessions whenever they want to. The disclosed embodiments present narrative information in an interactive format, with instructional guidance and/or via human-machine dialogue with/without an avatar rather than articles. The disclosed embodiments employ significantly better, more understandable visualizations of the data, including tagging which is integrated with visualizations so the user has a better sense of the factors that may be influencing the symptoms of OCD, social phobia, and other relative conditions.

IL should be appreciated that the examples set forth herein are provided merely for explanation and are in no way to be construed as limiting. While reference to various embodiments is made, the words used herein are words of description and illustration, rather than words of limitation. Further, although reference to special meaning, materials and embodiments are shown, there is no limitation to the particulars disclosed herein. Rather, the embodiments extend to all functionally equivalent structures, methods, and uses, such as are within the scope of the appended claims.

### Concepts

| | | | | Query |
|---|---|---|---|---|
| Name | Image | Sections | Count | match |
| Artificial | | title, abstract, | 8 | 0.000 |
| Intelligence-based | | description, claims | | |
| Anxiety disorder | | abstract, description, | 20 | 0.000 |
| | | claims | | |
| Avatar | | abstract, description, | 5 | 0.000 |
| | | claims | | |
| cognitive | | title, abstract, | 18 | 0.000 |
| | | description, claims | | |
| Cognitive-coping | | title, abstract, | 109 | 0.000 |
| therapy | | description, claims | | |
| Computer | | description, claims | 12 | 0.000 |
| Coping strategies | | abstract, description, | 10 | 0.000 |
| | | claims | | |
| Fear | | description, claims | 19 | 0.000 |
| Human-machine | | abstract, description, | 5 | 0.000 |
| dialogue | | claims | | |
| Machine learning | | Description | 1 | 0.000 |
| algorithms | | | | |
| Mental disorder | | abstract, description, | 13 | 0.000 |
| | | claims | | |
| Natural language | | abstract, description, | 4 | 0.000 |
| processing | | claims | | |
| Negative event | | description, claims | 11 | 0.000 |
| Network | | description, claims | 6 | 0.000 |
| Obsessive- | | abstract, description, | 47 | 0.000 |
| compulsive disorder | | claims | | |
| Panic disorder | | abstract, description, | 9 | 0.000 |
| | | claims | | |
| Smartphone | | description, claims | 4 | 0.000 |
| Social phobia | | abstract, description, | 11 | 0.000 |
| | | claims | | |
| Wearable device | | description, claims | 12 | 0.000 |

1. A computer-implemented method for implementing AI-based and non-AI-based cognitive-coping therapy (CCT) for a patient using NLP, Artificial Intelligence Language Model (AILM) tools or other generative AI products, and/or human-machine dialogue with/without an avatar, said method comprising: inputting a patient profile via an application executing on a processor of a patient device (computer, smartphone, wearable), said application being adapted to input data directly from the patient and from one or more wearable electronic devices associated with the patient; analyzing the profile to generate a CCT plan and process treatment sessions of CCT for the patient with mental disorders; monitoring patient data and other input data obtained as the patient implements the CCT plan and the treatment sessions to determine a status of the CCT plan; and determining if the CCT requires adjustment based on the monitored patient data and other input data.

2. The method according to claim 1, wherein one or more wearable electronic devices comprise one or more health monitors, exercise monitors, obsession monitors, fear of negative event monitors, anxiety monitors, or compulsion monitors.

3. The method according to claim 1, wherein treatment strategies focus on the fear of negative event(s) for treating OCD or anxiety disorder and focus on using coping strategies to divert the patient's attention from the fear, obsessions/compulsions, and/or anxiety to daily life and social functioning.

4. The method according to claim 1, wherein future stages of the CCT plan can be analyzed, adjusted, or skipped based on inputs from the patient or data from one or more wearable electronic devices associated with the patient.

5. The method according to claim 1, wherein information used to generate and monitor the CCT plan is received from a server computer over a network connection.

6. The method according to claim 1, wherein information used to generate and monitor the CCT plan is received from one of a camera or accelerometer of the patient device.

7. The method according to claim 1, wherein information from the application is transmitted from the patient's device to a cloud-based health monitoring system.

8. The method according to claim 1, further comprises reporting the status of how the patient is progressing through the CCT plan via one of the messages or graphical illustrations of one or more parameters of the CCT plan.

9. The method according to claim 1, wherein the application provides a log function and information input into the log is used to assess the status of the CCT plan.

10. The method according to claim 1, wherein the application comprises one or more of a coping portion adapted to educate the patient about enhancing coping strategies, a cognitive portion adapted to address unhelpful thinking or obsessions, a maintenance portion adapted to create a personalized reference and provide tips for handling lapses or an electronic bookshelf portion adapted to provide a central location for reference information provided by the application.

11. The method according to claim 1, wherein the CCT plan is used to treat mental disorders, like OCD, anxiety disorder (social phobia or panic disorder), and more.

12. A system for implementing CCT for treating a patient, said system comprising: an application program to be executed by a processor of a patient device, said application being adapted to: input a patient profile by inputting data directly from the patient and one or more wearable electronic devices associated with the patient; analyze the profile to generate a CCT plan for the patient; monitor patient data and other input data obtained as the patient implements the CCT plan to determine a status of the CCT plan; and determine if the CCT requires adjustment based on the monitored patient data and other input data.

13. The system according to claim 12, wherein one or more wearable electronic devices comprise one or more health monitors, exercise monitors, obsession monitors, anxiety monitors, or compulsion monitors.

14. The system according to claim 12, wherein future stages of the CCT plan can be analyzed, adjusted, or skipped based on the patient's inputs or data from wearable electronic devices.

15. The system according to claim 12, further comprises a server computer, wherein information used to generate and monitor the CCT plan is received from the server computer over a network connection.

16. The system according to claim 12, wherein information from the application is transmitted from the patient's device to a cloud-based health monitoring system.

17. The system according to claim 12, wherein the application is further adapted to report the status of how the patient is progressing through the CCT plan via one of messages or graphical illustrations of one or more parameters of the CCT plan.

18. The system according to claim 12, wherein the application provides a log function and information input into the log is used to assess the status of the CCT plan.

19. The system according to claim 12, wherein the application comprises one or more of a coping portion adapted to educate the patient about enhancing coping strategies, a cognitive portion adapted to address unhelpful thinking, a maintenance portion adapted to create a personalized reference and provide tips for handling lapses or an electronic bookshelf portion adapted to provide a central location for reference information provided by the application.

20. The system according to claim 12, wherein the CCT plan is used to treat obsessive-compulsive disorder, anxiety disorder (such as social phobia or panic disorder), and/or other mental disorders.
